(19) United States
(12) Patent Application Publication
(10) Pub. No.: US 2025/0037846 A1
(43) Pub. Date: Jan. 30, 2025

(54) SYSTEMS AND METHODS FOR GENERATING MEDIA MIX MODELS

(71) Applicant: MEDIDATA SOLUTIONS, INC., New York, NY (US)

(72) Inventors: Hartaig Singh, Plainview, NY (US); Eric Yang, Warminster, PA (US); Vikas Hiremath, Brooklyn, NY (US); Hugo Cacha, Lima (PE)

(21) Appl. No.: 18/361,388

(22) Filed: Jul. 28, 2023

(51) Int. Cl. G16H 40/20; G06N 20/20

(52) U.S. Cl. CPC G16H 40/20; G06N 20/20

(57) ABSTRACT

Disclosed are methods and systems for generating a media mix model. A time series data set is received specifying media delivered to recipients via a plurality of media channels at a plurality of times and one or more responses at the plurality of times. A random forest model is trained, the random forest splitting the time series data into subsets based on media channel of the plurality of media channels. Response curves are generated using the trained random forest model, each of the response curves corresponding to a media channel of the plurality of media channels, the response curves forming a media mix model adapted to predict responses based on media delivered and media channel.

### BACKGROUND

### Technical Field

The present disclosure generally relates to generating media mix models, and, in particular, media mix models adapted to predict responses based on media delivered and media channel.

### Description of the Related Art

It is difficult for organizations to analyze the impact that online ad campaigns have on sales. Conventional approaches to analysis in this field have suffered from the fact that they require significant amounts of additional research and knowledge of the impact of media. The models used in conventional approaches typically rely on Bayesian regressions. However, given the large number of independent channels (e.g., different keywords, platforms, etc.), it can be difficult to implement Bayesian approaches.

Among other things, conventional Bayesian approaches require a large number of parameters to be guessed and suffer from technical challenges, such as collinearity and lack of out-of-the-box non-linearity and interaction effects. To overcome these shortcomings, Bayesian models typically require additional components, such as Hill transformations, adstocking, interaction and mixed effect terms, which significantly increases the number of parameters requiring pre-existing knowledge and/or understanding. Incorrectly specifying such parameters can have a significant impact on insight, while, in many cases, not providing an appreciable difference in the quality of the underlying regression curve fits. Therefore, modelers must intervene and manually determine and/or adjust parameters, which effectively results in a process which is not data driven or model-based. All of this can result in downstream inefficiencies and significant, time-consuming manual effort.

An example of a conventional approach using Bayesian regression is discussed in the following research paper, which outlines a modeling process and the requirements of the multitude of parameters to be determined: “Bayesian Methods for Media Mix Modeling with Carryover and Shape Effects,” Yuxue Jin, Yueqing Wang, Yunting Sun, David Chan, Jim Koehler, Google Inc. (Apr. 14, 2017).

### SUMMARY

Disclosed embodiments relate to media analytics, media mix modeling, and promotion response. One use case is pharmaceutical media optimization, but the disclosed approaches can be used by any entity that serves media and/or promotional activity and would like to optimize media spend and/or quantitatively assess the impact that promotional activity has on users. Additionally, the framework described herein can also be used to identify the most impactful audiences to target and the effectiveness of different channels compared to each other.

Disclosed embodiments use tree-based approaches to compute response curves of media channels for specific media campaigns. Tree-based models capture non-linearity, interplay between variables, handle large amounts of features effectively, and require little or no assumptions and/or speculation regarding the parameters which are to be estimated, e.g., parameters relating to impact and diminishing returns of media and/or promotional activity along with optimal spend allocation. The disclosed tree-based models are more accurate, faster, data-driven, scalable, and significantly less time consuming than conventional Bayesian regression approaches. Furthermore, with tree-based modeling mixes, the models can be run while only specifying a handful of parameters, such as, for example: number of trees, tree depth, and lookback lengths. Thus, the disclosed approaches eliminate many of the pitfalls of conventional approaches.

In one aspect, the disclosed embodiments provide methods, systems, and computer-readable media for generating a media mix model. The methods include receiving a time series data set specifying media delivered to recipients via a plurality of media channels at a plurality of times and one or more responses at the plurality of times. The methods further include training a random forest model, the random forest splitting the time series data into subsets based on media channel of the plurality of media channels. The methods further include generating response curves using the trained random forest model, each of the response curves corresponding to a media channel of the plurality of media channels, the response curves forming a media mix model adapted to predict responses based on media delivered and media channel.

Embodiments may include one or more of the following features, alone or in combination.

The recipients may include health care providers in a plurality of defined specialties. The response curves may be specific to health care provider specialty. The responses may include at least one of: sales values and prescription quantities. The recipients may include patients and the responses may correspond to quantity of prescriptions filled. The plurality of media channels may include at least two of: emails, phone calls, and digital engagements. The methods may include specifying a lookback parameter defining a lag in the one or more responses.

### BRIEF DESCRIPTION OF THE DRAWINGS

FIG. 1 depicts a system including a Media Mix Modeling Subsystem adapted to receive data in the form of time series data of media and responses, according to disclosed embodiments.

FIG. 2 is a diagram depicting a random forest model which is trained with time series data of media and responses.

FIG. 3 depicts an example of a response curve for a digital media channel determined by random forest prediction, based on simulated data.

FIGS. 4-6 depict examples of response curves for a digital media channel, for particular health care provider (HCP) specialties, determined using a random forest model compared to four different Bayesian models for a set of simulated data.

FIG. 7 is a table summarizing the results of analysis using a simulated data set to compare the disclosed random forest model with four different Bayesian models.

FIG. 8 is a table summarizing the model components for the random forest model compared to the Bayesian models.

Where considered appropriate, reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth in order to provide a thorough understanding of embodiments of the invention. However, it will be understood by those of ordinary skill in the art that the embodiments of the present invention may be practiced without these specific details. In other instances, well-known methods, procedures, components, and circuits have not been described in detail so as not to obscure the present invention.

Media analytics involves measuring, managing, and analyzing market performance to maximize effectiveness and optimize return on investment (ROI). In the context of pharmaceutical marketing, this may involve tracking metrics related to sales force effectiveness, patient adherence to medication regimens, the effectiveness of direct-to-consumer advertising, or the response to different digital media campaigns. By tracking these metrics, pharmaceutical companies can identify what is working and what is not, and then adjust their media strategies accordingly. This data-driven approach helps to reduce wasted spending and focus efforts on the most impactful media initiatives.

Media mix modeling (or “marketing mix modeling”) is a statistical technique that uses historical data to quantify the impact of various media tactics on sales. The goal is to understand the effectiveness of each media tactic in the overall “mix,” to allocate media resources more efficiently. For a pharmaceutical company, this may involve analyzing how factors such as sales force effort, direct-to-patient advertising, physician detailing, sampling, online media, events and sponsorship etc. impact sales of a particular drug. Media mix modeling may involve running regression models with sales as the dependent variable and various media inputs as independent variables. The resulting coefficients provide estimates of the impact of each media input on sales. For example, if the model finds that physician detailing has a particularly strong impact on sales, the company might choose to allocate more resources to that area.

Promotion response analysis is an aspect of media analytics that specifically focuses on understanding how recipients, e.g., health care providers and/or patients, respond to various promotional activities. In the context of pharmaceutical marketing, this might involve analyzing how doctors, hospitals, and/or patients respond to different types of promotions, such as price discounts, product samples, or educational events. By understanding how different stakeholders respond to different types of promotions, pharmaceutical companies can optimize their promotional strategies to drive the highest possible response. For instance, if data analysis shows that hospitals are particularly responsive to educational events, a pharmaceutical company might choose to allocate more of its promotional spend to organizing such events.

Using one or more of these techniques—media analytics, media mix modeling, and promotion response analysis—can provide pharmaceutical companies with a robust framework for optimizing their media efforts. First, media analytics provides a general overview of the effectiveness of various media strategies. Then, media mix modeling dives deeper into the data to quantify the impact of each strategy. Lastly, promotion response analysis helps to optimize promotional activities. Taken together, these techniques can help pharmaceutical companies to allocate their media resources in the most effective and efficient way, driving better sales performance and a higher return on investment.

Pharmaceutical media optimization, which is a particular form media mix modeling, is a complex technical problem that can significantly benefit from technical solutions for several reasons:

Volume and Variety of Data: Pharmaceutical companies have access to vast amounts of data, from sales and media data to patient demographics and disease prevalence. Processing and making sense of such diverse and large-scale data manually is nearly impossible and prone to error. A technical solution can handle these large data sets and find patterns or insights that might not be evident through manual analysis.

Need for Precision: Accurate allocation of media resources can have a significant impact on a pharmaceutical company's bottom line. Misallocation of resources or incorrect assessments of media campaigns can result in significant lost revenue. Technical solutions, such as machine learning algorithms, can analyze complex data sets and provide precise recommendations for optimizing media strategies.

Complex Relationships: The relationship between media inputs and outputs is often nonlinear and may involve complex interactions and time lags. For example, the effect of a TV advertisement might be different when combined with a digital media campaign or might take time to materialize. Unraveling these relationships requires sophisticated statistical and machine learning models.

Dynamic Environment: The pharmaceutical industry operates in a dynamic environment, with changing regulations, competitive landscape, and market conditions. Therefore, media strategies need to be regularly updated and optimized based on the latest data. This requires a technical solution that can continuously learn from new data and adapt to changes.

Scalability: As pharmaceutical companies expand their products and markets, the complexity of media optimization increases exponentially. Technical solutions can scale to handle this complexity and provide actionable insights across different products and markets.

Thus, human expertise and judgment need to be complemented by technical solutions to effectively tackle the challenges of pharmaceutical media optimization.

The disclosed embodiments provide a technical solution which, inter alia, improves efficiency by significantly reducing the time and effort required to achieve spend optimization and promotional response estimates along with more accurate insights. The disclosed embodiments accomplish this without requiring pre-specification of a multitude of parameters, as in some conventional approaches, thereby reducing the risk of incorrect specification of important parameters.

The technical approaches described herein provide a process which can be scaled and automated more efficiently, which, in turn, allows the process to be data-driven, as opposed to conventional approaches which often require significant degrees of human intervention.

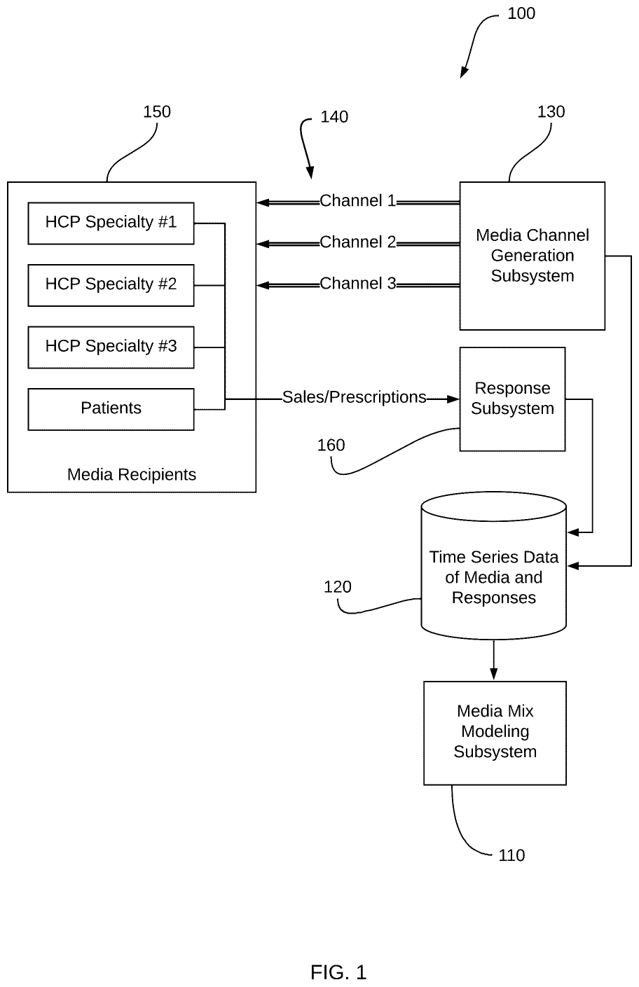

FIG. 1 depicts a system 100 including a Media Mix Modeling Subsystem 110 adapted to receive data in the form of time series data of media and responses 120. The time series data 120 is a combination of data from a Media Channel Generation Subsystem 130, which generates media channels 140—identified as Channel 1, Channel 2, and Channel 3 in this example. In embodiments, Channel 1 may correspond to emails, Chanel 2 may correspond to phone calls, and Channel 3 may correspond to digital (e.g., digital advertisements and engagement by social media and/or search engine). The media channels 140 are delivered to media recipients 150, which may include health care providers (HCP)—as a whole or considered by specialty—and/or patients. The system 100 further includes a Response Subsystem 160, which receives response data from the media recipients 150, which may be data relating to sales and/or number of prescriptions. In embodiments, the Media Mix Modeling Subsystem 110, Media Channel Generation Subsystem 130, and Response Subsystem 160 may be implemented on one or more computer systems comprising processors and memory.

Biotech and pharmaceutical companies, as noted above, have access to substantial amounts of data regarding media channel usage and sales, e.g., in terms of sales and/or number of prescriptions. Media channels 140 may include various forms of communication between the company (i.e., the company responsible for the media channels) and the health care providers (HCP) and/or patients. In terms of media mix modeling, the HCP is the recipient or “consumer.” Alternatively, or in addition, the data may be based on patient activity, e.g., prescriptions filled versus media channels directed to patients, in which case the patient is the recipient for purposes of analysis. This data may include sales information according to HCP specialty, e.g., neurology, oncology, hematology, etc.

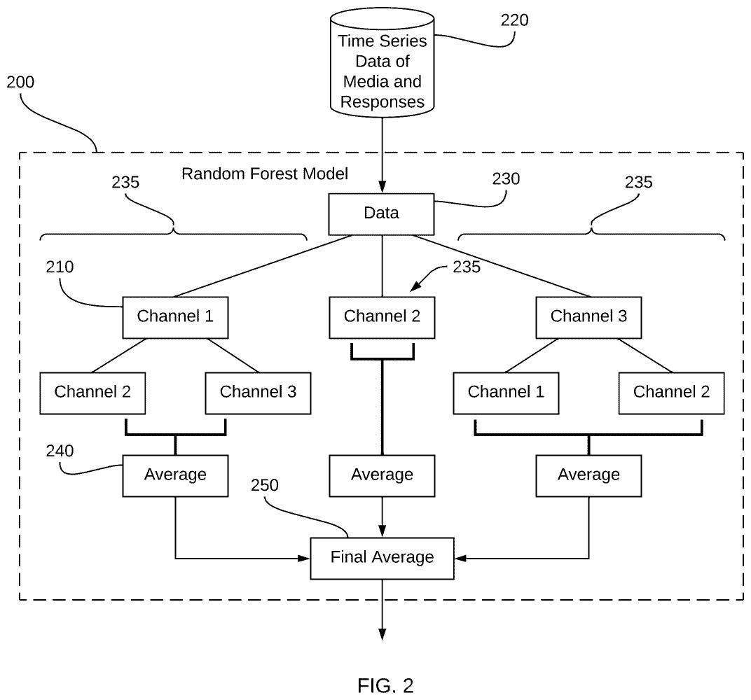

FIG. 2 is a diagram depicting a random forest model 200 which is trained with time series data of media and responses 220. A random forest is an ensemble model in which many decision trees are built on different subsets of the data and their predictions are averaged to get a final result. The diagram shows only a few nodes 210 of the model, whereas there may be, for example, 100 decision trees 235 in a typical model, each including one or more nodes 210. The input to the model is a time series data set 220, which is received by a first node 230 representing the entire data set. The data set is split into subsets, e.g., based on media channel, to be processed by separate trees 235. The results of particular nodes and/or combinations of nodes forming each tree 235 are received by an averaging node 240, and a final average result for the model is determined at a final averaging node 250 based on the averages for the individual trees 235, and this value is output by the model.

The data set may be based on a monthly time interval and may include, for example, the number of prescriptions issued per month per health care provider specialty (i.e., the response to be predicted by the trained model). The data set further includes media channel data, such as the number of phone calls, emails, and digital advertisements for each corresponding record of the dataset (i.e., the independent variables to be input to the trained model).

It should be noted that, unlike conventional regressions, the random forest model 200 is capable of handling a large variety of variables and their relationships. This capability allows for the inclusion of multiple non-media variables and their complex relationships to prescriptions and/or sales, which, in turn, more accurately estimates the impact of media and is more reflective of reality. Without the inclusion of such variables, models tend to overestimate the impact of media. An example of such a variable would be health care provider (HCP) specialty, as used in the simulated data set discussed below. Other examples include: inflation, region, COVID case count, formulary status, health care organization setting, etc. In implementations, the random forest model 200 could readily be extended to include more trees and nodes as the variable space increases.

Thus, the model, in effect, takes different splits of the data based on particular variables and then continues to process further subdivisions of the data set. The model seeks to maximize whichever of these branches or trees lead to the minimum difference between the result of the model (i.e., the predicted dependent variable) and the actual results (e.g., the actual values from the training data set). In other words, the model optimizes the path to the most accurate prediction. In doing so, the model accounts for interplay between channels as well. For example, if media channel 1 corresponds to the number of phone calls and media channel 2 corresponds to the number of emails, a node of the model may split at a threshold of, e.g., over 100 phone calls if that value is determined to be predictive. A branch of the split representing over 100 phone calls may be received by a node that splits based on channel 2, the number of emails, thus establishing a path involving interdependence between phone calls and emails.

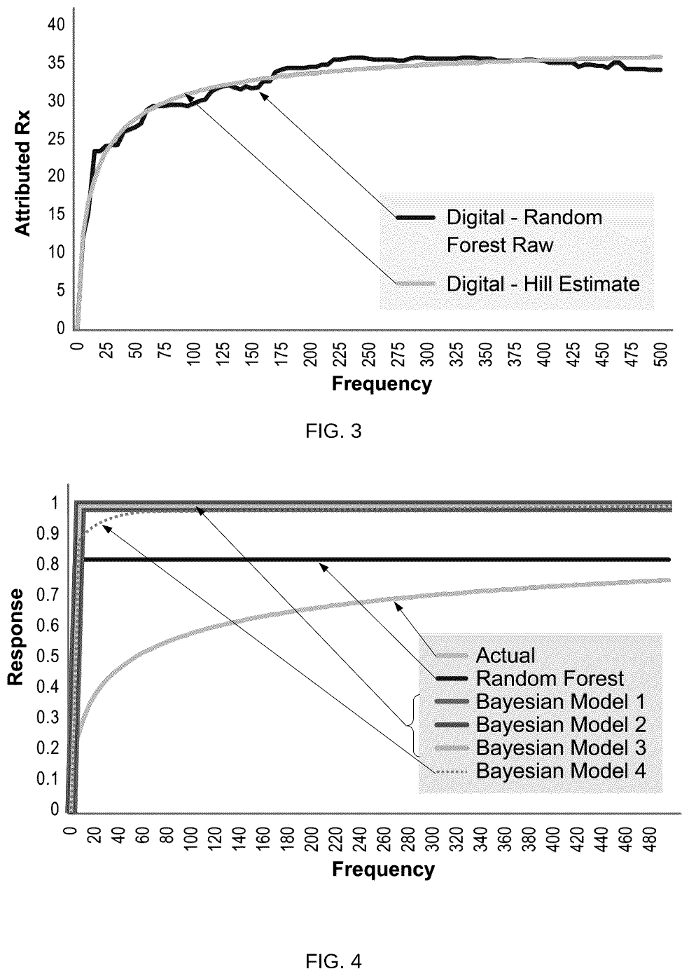

FIG. 3 depicts an example of a response curve for a digital media channel determined by random forest prediction, based on simulated data. The goal of the analysis using simulated data was to test the efficiency of random forests over traditional Bayesian regressions. This was tested by observing the following characteristics of each model: ability to capture saturation via response curves; number of assumptions and/or judgement calls required; number of parameters needed to define the model, predictive and curve fit performance; and flexibility with prior information.

The ragged curve corresponds to average predictions for each frequency minus the predictions when the digital channel equals zero, where frequency corresponds to a specific number of contacts via the digital channel during a defined period, e.g., one month. The smooth curve is a Hill function which has been fitted onto the digital channel response curve. A response curve may be parameterized in this manner for use in further calculations.

In disclosed embodiments, and based on analysis of simulated data, it is shown that models based on random forest approaches capture non-linearity in response curves, are better at handling a large number of features and collinearity, and identify interplay between media channels without the interplay having to be explicitly specified by the model designer. This removes the need to manually define forced saturation transformations and minimize the danger of misspecifying interactions between different channels. These advantages aid in reducing model complexity and modeler discretion, enabling more granular level insight, scalability, and automation.

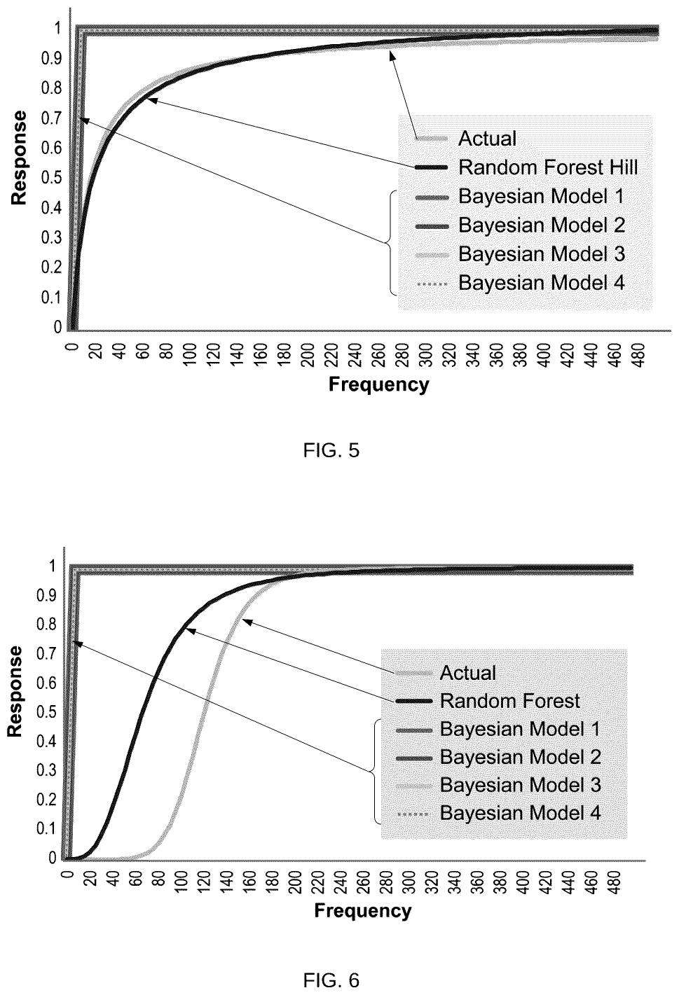

FIGS. 4-6 depict examples of response curves for a digital media channel, for particular health care provider (HCP) specialties, determined using a random forest model compared to four different Bayesian models for a set of simulated data.

Bayesian regressions combined with nonlinear transformations and adstock decay are typical approaches to building media mix models. However, such approaches require significant discretion and judgement on the part of the model designer, especially with respect to the “priors.” This tends to make it difficult to scale such models in a practical manner.

Priors, as used in Bayesian models, represent existing beliefs about the parameters in the model before observing the data. In the case of media mix modeling, a prior might represent an existing belief about how much a certain type of advertising (e.g., email or digital) influences sales. For example, if there is historical evidence or expert opinion suggesting that digital advertising has a large effect on sales, a prior may be set that reflects this belief. Bayesian regression models then update these priors with data to obtain posterior estimates of the parameters, which represent updated beliefs after observing the data. However, setting priors can be subjective and requires careful consideration. Inappropriate priors can bias the results and lead to over-estimation or underestimation of the effects of a particular media channel on sales or, in examples discussed herein, prescriptions written.

The smooth curve on each plot represents the actual response. The response curves were normalized to a standard scale. The simulated data was historical month-level response data per specialty from a two-year period. In implementations, the data can be structured according to any level of interest (e.g., HCP specialty—week, zipcode—month, hospital—month, etc.). The simulated data, thus, defines a time series, the response (e.g., prescription count), and the number of contacts for each media channel, which in the present example are email, phone, and digital (e.g., digital advertisements delivered by a search engine or social media platform). In implementations, various key performance indicators (KPI), e.g., sales, can be used as a basis for analysis.

FIG. 4 depicts the digital channel response curves for neurologists. The impact of the digital channel on neurologists was set to be low, relative to the other specialties, to assess the performance of the models for low importance, i.e., low signal, channels. All of the models performed poorly under these constraints. However, it may not be necessary in practice to reach low-impact audiences.

FIG. 5 depicts the digital channel response curves for hematologists, and FIG. 6 depicts the digital channel response curves for oncologists. The smooth curve on each plot represents the actual response. In contrast to the example of neurologists, discussed above, these plots show that the response curves generated using the random forest model fit the actual response curves rather closely. The fit of the random forest model was better for hematologists (FIG. 5) than for oncologists (FIG. 6) in this simulated example, because the hematologist specialty was defined in the simulated data to be a dependent variable having more importance, i.e., a stronger signal, relative to the other specialties. In practice, this means that the importance of a dependent variable is a good indicator that the response curve produced using the random forest model is accurate.

FIGS. 4-6 show that the Bayesian models did poorly for all three specialties. This may be due to misspecification of priors, as these have a significant impact on the performance of Bayesian models. Although the comparison presented in FIGS. 4-6 are based on simulated data, it is apparent that it is difficult to set up Bayesian models to perform these functions even under simulated conditions.

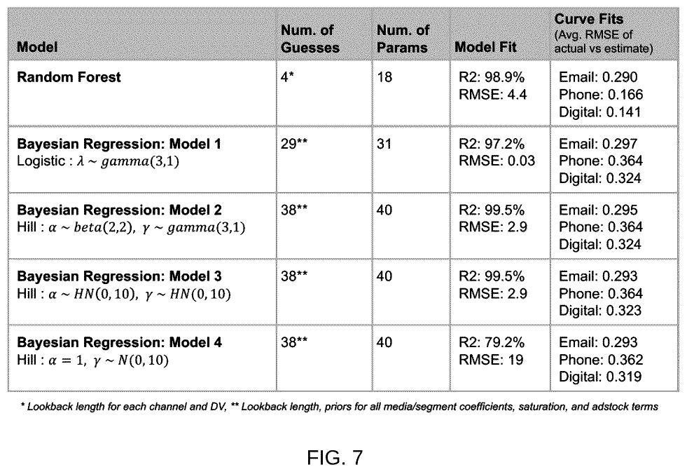

In the simulation, it was found that varying the prior distribution resulted in similar estimated response curves, showing the need to carefully specify and define informative priors. FIG. 4, as noted above, shows that model performance is not always related to true underlying promotional response. FIGS. 5 and 6 and the table of results (FIG. 7, discussed below) show the ability of random forest models to capture segment-level responses and estimate true fits significantly better than Bayesian models.

Thus, the random forest model achieves more reasonable promotion response fits while requiring the specification of only a relatively small set of parameters. Especially in cases where only minimal and/or weak prior information is available, random forest models may be a stronger first attempt than a Bayesian model.

FIG. 7 is a table summarizing the results of analysis using a simulated data set to compare the disclosed random forest model with four different Bayesian models. The simulated data set provided data for health care provider specialties and, in particular, month-level data for three media channels (i.e., email, phone, and digital) using specified Hill parameters to force c-shaped and s-shaped response curves. The data set contained three different specialties (i.e., segments), each with 120 time points. The dependent variable was the sum of the outputs for each Hill equation across each segment.

A random forest model, as in the disclosed embodiments, and four different Bayesians regression models were specified. For the purposes of this analysis, no hyperparameter tuning was performed on the random forest model (i.e., default sklearn random forest regressor hyperparameters were used). The prior assumptions of the saturation parameter were varied for each regression. All of the models were designed to capture carryover and saturation effects by segments. Because the simulation was specified, true segment-level responses and parameters were available for comparison to the estimated response curves of the models, as summarized in the table of FIG. 7.

The table of FIG. 7 provides an assessment of the random forest model versus the four Bayesian models in terms of R-squared (R2) and root mean square error (RMSE). Some of the Bayesian have a slightly higher R2 than the random forest model because they could have slightly better predictions. However, the more pertinent characteristic is curve fit, as indicated by the average RMSE between the actual response curve of each channel and the estimated response curve across all specialties (see “Curve Fits” column of table). In every case, the random forest model has lower channel RMSE, which means that it provides a better estimate of the response curves than the Bayesian regressions. In general, an R-squared value measures the proportion of the variance in the dependent variable that can be predicted from the independent variables. It ranges between 0 and 1, with a value closer to 1 indicating a better fit of the model (the R-squared values are expressed as a percentage in the table, where a value closer to 100% indicates a better fit of the model). However, as noted above, because the primary goal of these models is to estimate accurate response curves, the overall model performance in terms of R2 is not especially relevant. For example, Bayesian Model 4 had the worst overall performance but the best curve fits among the Bayesian regressions. Also, it should be noted that a high R-squared value does not necessarily mean the model is good, and it can even be artificially high if the model is overfitted.

For purposes of comparison, the number of parameters specified in the table of FIG. 7 for the random forest model includes the number of variables in the model, i.e., there are 18 features in the model, in total. Included in the 18 parameters for the random forest model are 4 guesses, which include a lookback parameter for each of the three media channels and the dependent variable (e.g., number of prescriptions written). For the Bayesian models, on the other hand, each feature is, strictly speaking, another coefficient.

The lookback parameter for the random forest model defines a lag in the dependent variables, e.g., the analysis is based on a previous month's sales or prescriptions. A decision has to be made as to how far to look back, so that the parameter can be tuned accordingly. A typical lookback value in this context would be, for example, three months. Another parameter to be tuned in the random forest model is the number of decision trees. It the simulation documented here, the number of trees was set to 100.

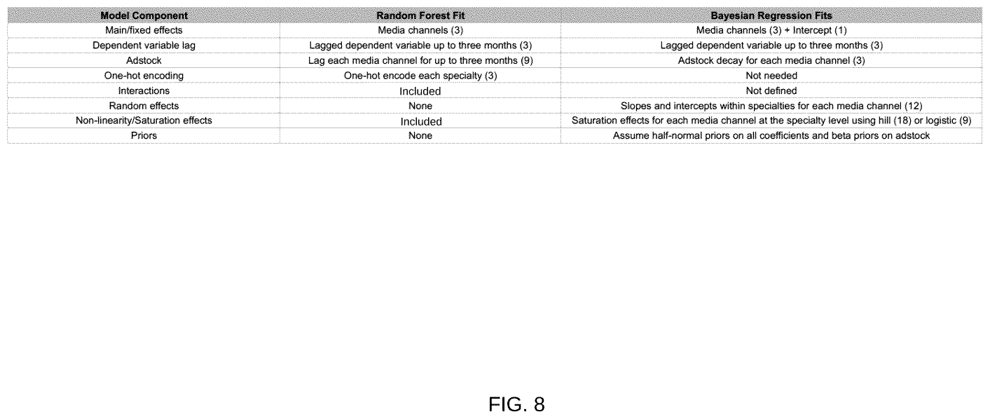

FIG. 8 is a table summarizing the model components for the random forest model compared to the Bayesian models. The numbers in parentheses indicate the number of parameters required by each component—the total being equal to the number of parameters specified in FIG. 7, discussed above. For example, for the main (i.e., fixed) effects, there were three dependent variables corresponding to the three media channels in the simulation and, for the Bayesian models, an intercept is defined (resulting in four parameters). As a further example, dependent variable lag may be applied for a defined period of time, e.g., three months, to account for seasonality. The dependent variable lag required three additional parameters for either the random forest or Bayesian models.

The Bayesian models required several priors to provide usable results. For example, a prior was needed to ensure a positive result, i.e., a result that is normalized. Also, because these models are linear, a curved characteristic must be imposed to ensure that there is a definitive saturation point. This is difficult to do in practice, because it is unknown and, ideally, the saturation point is meant to be derived from the data itself. The random forest model, on the other hand, captures the non-linear behavior based on actual inflection points that are being provided by the data, thereby avoiding the need to pass in these priors to fix an inflection point on the saturation curve.

Additional parameters were required to effect an adstock delay, which operates on the independent variable. The adstock delay in a Bayesian model may be implemented by transforming a current feature by taking previous values, fractions of those, and adding it into the current feature, rather than by creating more features. In random forest models, it is possible to bypass adstock by just lagging the lagging the media channel variable (i.e., the independent variable) in a manner similar to the delay applied to the dependent variable. In this way, an adstock can be avoided in the random forest model. It should be noted that 9 parameters are used in implementing adstock for the random forest model because each channel is lagged up to 3 months and each of the lagged channels is included as a feature in the model. The implementation of adstock in the Bayesian model uses a specific adstock function that applies a geometric decay on each of the 3 channels. Therefore, an optimal decay parameter for each channel is estimated. An advantage of avoiding the adstock in the random forest model is that provide improved computational efficiency and scalability, because it removes the need to apply a function with a parameter that requires a prior.

As can be seen from the table of FIG. 8, in the areas of: (i) random effects, and (ii) non-linearity and saturation effects, the Bayesian models require numerous parameters to define, respectively: (a) slopes and intercepts within specialties for each media channel, and (b) saturation effects for each media channel at the specialty level using Hill or logistic curves. This is where the random forest model has a significant advantage, as these parameters are either not needed or already included in the model. Thus, the number of parameters to be guessed and the total number of parameters for the random forest model are substantially less than in the Bayesian models.

The simulation results showed that the random forest model, in addition to capturing the non-linear behavior very well, also provided a much better fit to the response curves (i.e., the actual response curves obtained from the simulated data set).

Another advantage of the random forest model is that it provides an indication of feature importance as an output. Feature importance, in effect, shows the impact on performance if a particular feature were removed from the data set. In the simulation, the feature importance was output as a value between zero and one, with a value of one corresponding to high importance. The higher the feature importance the stronger the fit of the response curve, which provides helpful insight, because normally one does not know how well a response curve fits to true behavior. Therefore, the feature importance, in effect, serves as a leading indicator of whether the response curve is accurate.

Aspects of the present invention may be embodied in the form of a system, a computer program product, or a method. Similarly, aspects of the present invention may be embodied as hardware, software, or a combination of both. Aspects of the present invention may be embodied as a computer program product saved on one or more computer-readable media in the form of computer-readable program code embodied thereon.

The computer-readable medium may be a computer-readable storage medium. A computer-readable storage medium may be, for example, an electronic, optical, magnetic, electromagnetic, infrared, or semiconductor system, apparatus, or device, or any combination thereof.

Computer program code in embodiments of the present invention may be written in any suitable programming and/or scripting language. The program code may execute on a single computer, or on a plurality of computers. The computer may include a processing unit in communication with a computer-usable medium, where the computer-usable medium contains a set of instructions, and where the processing unit is designed to carry out the set of instructions, and/or a trained machine learning algorithm. The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A method of generating a media mix model, the method comprising:
receiving a time series data set specifying media delivered to recipients via a plurality of media channels at a plurality of times and one or more responses at the plurality of times;
training a random forest model, the random forest splitting the time series data into subsets based on media channel of the plurality of media channels; and
generating response curves using the trained random forest model, each of the response curves corresponding to a media channel of the plurality of media channels, the response curves forming a media mix model adapted to predict responses based on media delivered and media channel.

2. The method of claim 1, wherein the recipients comprise health care providers in a plurality of defined specialties.

3. The method of claim 2, wherein the response curves are specific to health care provider specialty.

4. The method of claim 1, wherein said one or more responses comprise at least one of: sales values and prescription quantities.

5. The method of claim 1, wherein the recipients comprise patients and said one or more responses correspond to quantity of prescriptions filled.

6. The method of claim 1, wherein the plurality of media channels comprises at least two of: emails, phone calls, and digital engagements.

7. The method of claim 1, further comprising specifying a lookback parameter defining a lag in the one or more responses.

8. A system for generating a media mix model, comprising:
a computer having one or more processors in communication with a memory, the memory storing instructions executable by said one or more processors to perform:
receiving a time series data set specifying media delivered to recipients via a plurality of media channels at a plurality of times and one or more responses at the plurality of times;
training a random forest model, the random forest splitting the time series data into subsets based on media channel of the plurality of media channels; and
generating response curves using the trained random forest model, each of the response curves corresponding to a media channel of the plurality of media channels, the response curves forming a media mix model adapted to predict responses based on media delivered and media channel.

9. The system of claim 8, wherein the recipients comprise health care providers in a plurality of defined specialties.

10. The system of claim 9, wherein the response curves are specific to health care provider specialty.

11. The system of claim 8, wherein said one or more responses comprise at least one of: sales values and prescription quantities.

12. The system of claim 8, wherein the recipients comprise patients and said one or more responses correspond to quantity of prescriptions filled.

13. The system of claim 8, wherein the plurality of media channels comprises at least two of: emails, phone calls, and digital engagements.

14. The system of claim 8, wherein the memory further stores instructions executable by said one or more processors to perform specifying a lookback parameter defining a lag in the one or more responses.

15. A non-transitory computer-readable medium storing instructions that, when executed by one or more processors of a computer, cause said one or more processors to perform a method of generating a media mix model, the method comprising:
receiving a time series data set specifying media delivered to recipients via a plurality of media channels at a plurality of times and one or more responses at the plurality of times;
training a random forest model, the random forest splitting the time series data into subsets based on media channel of the plurality of media channels; and
generating response curves using the trained random forest model, each of the response curves corresponding to a media channel of the plurality of media channels, the response curves forming a media mix model adapted to predict responses based on media delivered and media channel.

16. The computer-readable medium of claim 15, wherein the response curves are specific to health care provider specialty.

17. The computer-readable medium of claim 15, wherein said one or more responses comprise at least one of: sales values and prescription quantities.

18. The computer-readable medium of claim 15, wherein the recipients comprise patients and said one or more responses correspond to quantity of prescriptions filled.

19. The computer-readable medium of claim 15, wherein the plurality of media channels comprises at least two of: emails, phone calls, and digital engagements.

20. The computer-readable medium of claim 15, wherein the method further comprises specifying a lookback parameter defining a lag in the one or more responses.
